(19) United States
(12) Patent Application Publication
(10) Pub. No.: US 2008/0172078 A1
(43) Pub. Date: Jul. 17, 2008

(54) REDUCED TRACTION VITRECTOMY PROBE

(76) Inventor: Ralph E. Svetic, Costa Mesa, CA (US)

Correspondence Address: ALCON, IP LEGAL, TB4-8, 6201 SOUTH FREEWAY, FORT WORTH, TX, 76134, US

(21) Appl. No.: 11/623,353

(22) Filed: Jan. 16, 2007

(51) Int. Cl. A61B 17/32

(52) U.S. Cl. 606/171

(57) ABSTRACT

A vitrectomy probe that minimizes retinal traction by balancing infusion inflow and aspiration outflow near its cutting port.

### FIELD OF THE INVENTION

The present invention generally pertains to microsurgical instruments. More particularly, but not by way of limitation, the present invention pertains to microsurgical instruments used in posterior segment ophthalmic surgery, such as vitrectomy probes.

### DESCRIPTION OF THE RELATED ART

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worst case, cutting and removal of the retina itself.

The use of microsurgical cutting probes in posterior segment ophthalmic surgery is well known. Such vitrectomy probes are typically inserted via an incision in the sclera near the pars plana. The surgeon may also insert other microsurgical instruments such as a fiber optic illuminator, an infusion cannula, or an aspiration probe during the posterior segment surgery. The surgeon performs the procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port, and the inner member is actuated, closing the port. Upon the closing of the port, cutting surfaces on both the inner and outer cutting members cooperate to cut the vitreous, and the cut vitreous is then aspirated away through the inner cutting member. U.S. Pat. No. 4,577,629 (Martinez); U.S. Pat. No. 5,019,035 (Missirlian et al.); U.S. Pat. No. 4,909,249 (Akkas et al.); U.S. Pat. No. 5,176,628 (Charles et al.); U.S. Pat. No. 5,047,008 (de Juan et al.); U.S. Pat. No. 4,696,298 (Higgins et al.); and U.S. Pat. No. 5,733,297 (Wang) all disclose various types of vitrectomy probes, and each of these patents is incorporated herein in its entirety by reference.

Conventional vitrectomy probes include “guillotine style” probes and rotational probes. A guillotine style probe has an inner cutting member that reciprocates along its longitudinal axis. A rotational probe has an inner cutting member that rotates about its longitudinal axis. In both types of probes, the inner cutting members are actuated using various methods. For example, the inner cutting member can be moved from the open port position to the closed port position by pneumatic pressure against a piston or diaphragm assembly that overcomes a mechanical spring. Upon removal of the pneumatic pressure, the spring returns the inner cutting member from the closed port position to the open port position. As another example, the inner cutting member can be moved from the open port position to the closed port position using a first source of pneumatic pressure, and then can be moved from the closed port position to the open port position using a second source of pneumatic pressure. As a further example, the inner cutting member can be electromechanically actuated between the open and closed port positions using a conventional rotating electric motor or a solenoid. U.S. Pat. No. 4,577,629 provides an example of a guillotine style, pneumatic piston/mechanical spring actuated probe. U.S. Pat. Nos. 4,909,249 and 5,019,035 disclose guillotine style, pneumatic diaphragm/mechanical spring actuated probes. U.S. Pat. No. 5,176,628 shows a rotational dual pneumatic drive probe.

In conventional vitrectromy probes and vitreoretinal surgeries, retinal traction remains a challenge for the surgeon and a potential safety issue for the patient. Typically, such traction results from the suction applied by the probe as it removes vitreous material cut from the eye. To maintain a safe intraocular pressure, an infusion cannula remotely supplies infusion fluid to the eye. However, there is still a net outflow of material at the tip of the probe that results in traction on the retina. Therefore, a need exists for an improved vitrectomy probe that reduces retinal traction and provides “cut on demand” tissue removal.

### SUMMARY OF THE INVENTION

In one aspect, the present invention is a vitrectomy probe having an engine and a distal end coupled to the engine. The distal end includes an outer cutting member with an opening for receiving tissue and an inner cutting member reciprocatingly disposed within the outer cutting member and fluidly coupled to a vacuum source. The outer member has a groove on its outer surface disposed parallel to a length of the outer cutting member and with an end of the groove terminating in the opening. A sleeve is disposed around the distal end and a proximal portion of the groove so as to create a lumen between the sleeve and the distal end. The lumen is fluidly coupled to an infusion source.

In another aspect, the present invention is a method of reducing retinal traction in vitreoretinal surgery. A vitrectomy probe is provided. The probe includes an engine and a distal end coupled to the engine. The distal end has an outer cutting member with an opening for receiving tissue and an inner cutting member reciprocatingly disposed within the outer cutting member. A sleeve is disposed around the distal end so as to create a lumen between the sleeve and the distal end. The inner cutting member is fluidly coupled to a vacuum source. The lumen is fluidly coupled to an infusion source. The inner cutting member is reciprocated within the outer cutting member. The infusion inflow from the lumen is balanced with the aspiration outflow through the inner cutting member proximate the opening when the opening is not engaging vitreoretinal tissue.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is a schematic, side, sectional view of a vitrectomy probe according to a preferred embodiment of the present invention;

FIG. 2 is an enlarged, side, sectional view of the distal end of the probe of FIG. 1; and

FIG. 3 is an enlarged, cross-sectional view of the distal end of the probe of FIG. 1 along line 3-3.

### DETAILED DESCRIPTION OF THE PREFERRED EMBODIMENTS

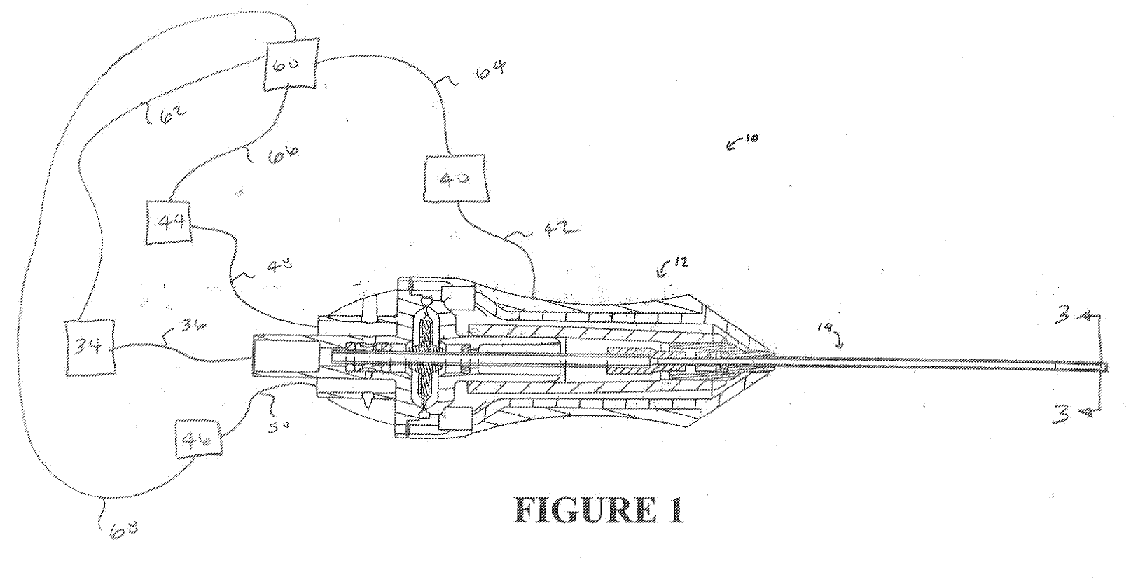

The preferred embodiments of the present invention and their advantages are best understood by referring to FIGS. 1-3 of the drawings, like numerals being used for like and corresponding parts of the various drawings.

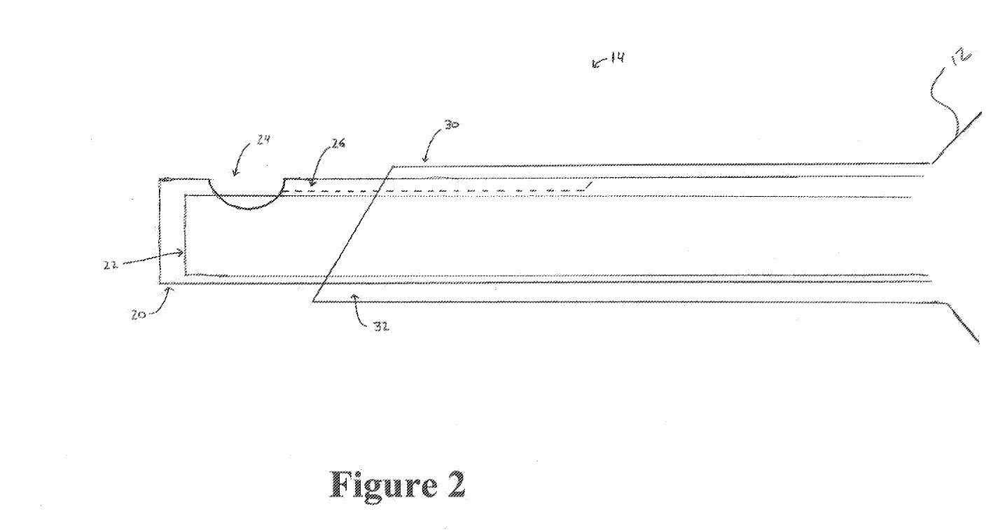

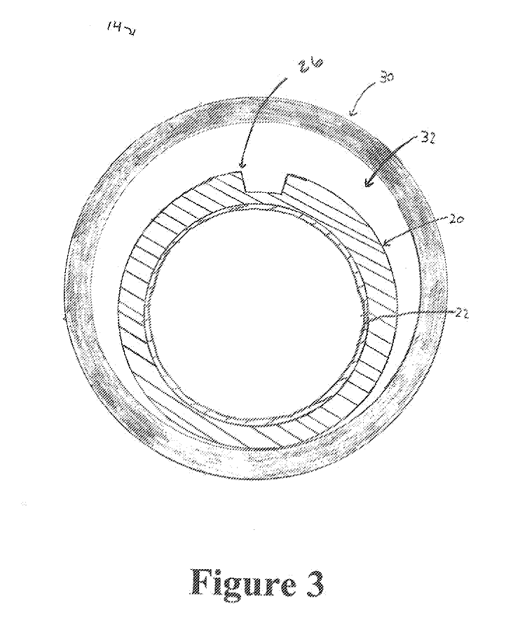

FIG. 1 illustrates that vitrectomy probe 10 generally includes an engine or body 12 and a distal end 14. FIGS. 2 and 3 show that distal end 14 comprises a hollow outer cutting member 20 with an opening or port 24 near its distal end. A notch 26 is disposed in the surface of outer cutting 20 beginning at opening 24 and extending proximally toward engine 12. Notch 26 is oriented to be parallel to the length of outer cutting member 20. A hollow inner cutting member 22 is disposed entirely within outer cutting member 20. Inner cutting member 22 is capable of reciprocating motion along the longitudinal axis of probe 10 within outer cutting member 20. Inner cutting member 22 is fluidly connected to a vacuum source 34 via tubing 36 and engine 12. Distal end 14 is disposed within a sleeve 30 in such a way that a lumen 32 is formed between the outer surface of outer cutting member 20 and the inner surface of sleeve 30. Lumen 32 is fluidly connected to an infusion source 40 via tubing 42 and engine 12. Sleeve 30 may be made from any appropriate material but is most preferably made from a lightweight material such as rubber or plastic. The remainder of distal end 14 is most preferably made from surgical stainless steel. Inner cutting member 22 may be driven by any conventional actuating means, but is most preferably driven by dual pneumatic pressure sources 44 and 46 that are fluidly coupled to engine 12 via tubing 48 and 50, respectively. A microprocessor or computer 60 is electrically coupled to vacuum source 34, infusion source 40, pneumatic pressure source 44, and pneumatic pressure source 46 via interfaces 62, 64, 66, and 68 respectively.

During operation, distal end 14 of probe 10 is inserted into the posterior segment of the eye via an incision in the sclera. Tissue enters outer cutting member 20 through opening 24 and is cut by the reciprocating inner cutting member 22. The cut material is aspirated through the hollow center of inner cutting member 22. To avoid retinal traction, an infusion fluid is delivered to the eye via lumen 32 in sleeve 30. Microprocessor 60 controls vacuum source 34, infusion source 40, and pneumatic pressure sources 44 and 46 so that the infusion inflow and aspiration outflow near opening 24 are balanced, resulting in no (or minimal) traction at the tip of probe 10. No cutting of tissue occurs when probe 10 is held in a static position. As cutting pressure is applied by the surgeon, the tip of probe 10 moves into vitreous material, and the vitreous material presses against and is drawn into opening 24 as the irrigation flow is diverted around the vitreous mass. Probe 10 will continue to cut and aspirate material only as long as such cutting pressure is maintained. When the cutting pressure is removed and probe 14 is again held in a static position, no cutting or traction occurs.

From the above, it may be appreciated that the present invention reduces retinal traction during vitreoretinal surgery. The present invention is illustrated herein by example, and various modifications may be made by a person of ordinary skill in the art. For example, although the present invention is described above in connection with a guillotine style, dual pneumatic vitrectomy probe, it is equally applicable to the other conventional vitrectomy probes described hereinabove.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A vitrectomy probe, comprising:
an engine;
a distal end coupled to said engine, said distal end comprising an outer cutting member with an opening for receiving tissue and an inner cutting member reciprocatingly disposed within said outer cutting member and for fluidly coupling to a vacuum source, said outer member having a groove on an outer surface disposed parallel to a length of said outer cutting member and with an end of said groove terminating in said opening; and
a sleeve disposed around said distal end and a proximal portion of said groove so as to create a lumen between said sleeve and said distal end, said lumen for fluidly coupling to a infusion source.

2. A method of reducing retinal traction in vitreoretinal surgery, comprising the steps of:
providing a vitrectomy probe, said probe comprising:
an engine;
a distal end coupled to said engine, said distal end comprising an outer cutting member with an opening for receiving tissue and an inner cutting member reciprocatingly disposed within said outer cutting member; and
a sleeve disposed around said distal end so as to create a lumen between said sleeve and said distal end;
fluidly coupling said inner cutting member to a vacuum source;
fluidly coupling said lumen to an infusion source;
reciprocating said inner cutting member within said outer cutting member; and
balancing an infusion inflow from said lumen with an aspiration outflow through said inner cutting member proximate said opening when said opening is not engaging vitreoretinal tissue.
